(19) United States
(12) Patent Application Publication
(10) Pub. No.: US 2017/0360720 A1
(43) Pub. Date: Dec. 21, 2017

(54) METHOD TO SUPPLEMENT HUMAN IMMUNE SYSTEM IN SUPPRESSING HSV SYMPTOMS

(71) Applicant: Melissa Pagen, Los Angeles, CA (US)

(72) Inventor: Melissa Pagen, Los Angeles, CA (US)

(21) Appl. No.: 15/625,030

(22) Filed: Jun. 16, 2017

Related U.S. Application Data
(60) Provisional application No. 62/351,484, filed on Jun. 17, 2016.

(51) Int. Cl. A61K 31/105; A61K 9/48; A61K 9/00; A61K 9/20; A61K 36/85; A61K 31/198

(52) U.S. Cl. CPC A61K 31/105; A61K 36/85; A61K 31/198; A61K 9/0053; A61K 9/20; A61K 9/48

(57) ABSTRACT

Some embodiments of the invention relate to methods of administering orally a therapeutically effective pharmaceutical formulation of between about 3,000 micrograms to about 12,000 micrograms of allicin in a pharmaceutical dosage form to a patient with a herpes simplex viral infection, the therapeutically effective pharmaceutical formulation of allicin for increasing a human immune system activity in the patient during a reactivation of a herpes simplex virus causing a prodromal phase of the herpes simplex virus outbreak in the patient, the increasing of the human immune system activity in the patient for suppressing a herpes simplex viral symptom in the patient. In a preferred embodiment of the invention, an initial allicin dose of between about 7,200 micrograms to about 12,000 micrograms is administered once to an HSV patient and then at hourly or daily intervals, nine allicin doses additional of between about 3,000 micrograms to about 4800 micrograms are administered to the HSV patient.

### CROSS-REFERENCE TO RELATED APPLICATIONS

This application claims the benefit of U.S. Provisional Patent Application No. 62/351,484 filed on Jun. 17, 2016 which is incorporated herein by reference.

### BACKGROUND OF THE INVENTION

Herpes Simplex Virus (HSV-1 and HSV-2), is one of the most common viruses affecting humans worldwide, with its first historical mention being most acceptably credited to Hippocrates of Kos (460 BC-370 BC), who was one of the most remarkable Greek physicians in the history of medicine. Although the word Herpes (meaning creeping) was arguably used as a broader term at this time in history, to describe skin lesions resulting from several different infections or diseases, and Hippocrates could have been referring to any one of these, its long-standing presence in human history is undisputed. Presently, HSV-1 and HSV-2 comprise the most closely related pair of herpes viruses for which complete genome sequences are known, but their genomes substantially diverged an estimated 8 million years ago' nonetheless. The DNA sequence of HSV 1 was only mapped as recently as 1988, and that sequence has since been revised. Mapping and publication of the DNA sequence of HSV 2 was only as recent as 1997. Yet, the most significant difference between type 1 and 2 is in the pathology—how each virus behaves in the body, for example viral reactivation, viral effect on cellular functions, and viral responsiveness to antiviral medication.

Despite the DNA sequence similarities and divergence on a molecular level, in medical practice and at the sociological level, the common distinction of HSV type 1 and 2 differentiates the region in which the virus has taken residence in the body and where symptoms are expressed as episodes or outbreaks. Outbreaks on the face from the virus taking residence in the trigeminal ganglion are commonly referred to as HSV 1. Outbreaks on the genitals are from the virus taking residence in the sacral ganglion are commonly referred to as HSV 2. While HSV 1 is commonly used to indicate the virus expressed on the face and HSV 2 is commonly referred to as the virus that is expressed on the genitals, HSV 1 and HSV 2 both can take residence in either location.

When HSV is contracted and takes residence in the trigeminal ganglion, the virus will typically express itself on the face. The trigeminal ganglion is a bundle of nerve cells of the trigeminal nerve, situated in the trigeminal cavity, located near the top of the temporal bone in a human head. The trigeminal nerve is responsible for sensation in the face and related motor functions, like biting or chewing. A ganglion is a cluster of neurons or nerve cells. Each nerve cell is comprised of a soma which is a cell body containing the cell nucleus, and dendrites which are branch-like structures that project from the soma. The virus resides dormant in the nerve cells in this region until it is triggered or reactivates, begins to replicate, and is carried to the face along the neuron's axon which is a projection from the nerve cell that carries electric pulses to the skin. The expression of a herpes simplex virus on the skin of a person is referred to as an episode or outbreak. Some of the widely accepted triggers of an episode are: anxiety, stress, sunburn, hormonal changes, and physical exhaustion. Typically, reactivation of the virus is accompanied by tingling, itching, or tenderness at the site of viral shedding. The sensations of tingling, itching and tenderness are called prodromes and occur during what is referred to as the prodromal phase. The active virus sheds on the surface of the skin of the face, usually in the area of the mouth (Herpes Labialis) and is highly contagious. In symptomatic hosts, vesicle formation occurs (‘wet blisters/cold sores’). The roof of each vesicle eventually collapses to form herpetic ulcers, which then scab until healed.

There are many misconceptions and misinformation about the virus, including conflicting opinions in the medical community surrounding its behavior, how and when it is contracted, and efficacy of various treatment methods. HSV is prolific. There is no cure for the virus, no way to prevent the expression of the virus on the body, and there is no current method to prevent the expression of symptoms of the virus on the face.

Current methods used to address the virus and/or its symptoms of herpes episode exist as anti-viral pharmaceutical prescriptions from a doctor and over the counter products. Prescription antiviral medicines (e.g. acyclovir [Zovirax], famciclovir [Famvir], and valacyclovir [Valtrex]) are prescribed for episodic treatment or suppressive treatment. Episodic treatment requires that the antiviral be taken when a reactivation and expression of the virus (outbreak) occurs and is no longer taken when the symptoms are gone. For the present invention, expression of the herpes virus means a herpes outbreak or a herpes episode. Suppressive treatment, generally recommended for those who experience frequent outbreaks, requires that the antiviral be taken daily to suppress the virus from reactivation. For the present invention, reactivation of the herpes virus means that the herpes virus is triggered by some event such as anxiety, stress, sunburn, hormonal changes, and physical exhaustion, or some unknown factor, and the virus begins to replicate, and the virus is carried to the skin of the face along the neuron's axon. Suppressive treatment with prescription antivirals is not 100% effective in suppressing viral reactivation. Antivirals used episodically or to suppress cannot stop future outbreaks, do not claim to prevent the expression of the virus, but claim to shorten the duration of the expression, or lessen the frequency in which outbreaks occur.

Not all who have the virus have access to prescription drugs. Paying regularly for a prescription used to treat a condition that will continue in perpetuity is not a cost that can be absorbed by all socioeconomic groups. As shown in the data below, there are millions affected in several developing countries. This worldwide data on the incidence of human herpes simplex viral infections in indicates that there is a great need for affordable, and available drug treatments of human herpes simplex viral infections.

Regional infection estimates

Estimates for HSV-1 prevalence by region among people aged 0-49 in
2012:
Americas: 178 million women (49%), 142 million men (39%)
Africa: 350 million women (87%), 355 million men (87%)
Eastern Mediterranean: 188 million women (75%), 202 million men (75%)
Europe: 207 million women (69%), 187 million men (61%)
South-East Asia: 432 million women (59%), 458 million men (58%)
Western Pacific: 488 million women (74%), 521 million men (73%)
Estimates of new HSV-1 infections among people aged 0-49 in 2012:
Americas: 6 million women, 5 million men
Africa: 17 million women, 18 million men
Eastern Mediterranean: 6 million women, 7 million men
Europe: 5 million women, 5 million men
South-East Asia: 13 million women, 14 million men
Western Pacific: 11 million women, 12 million men

With the increase in pursuits of healthy living, coupled with the intention of decreasing consumption/exposure to chemicals, and widespread notions of pharmaceutical companies having hidden agendas, there is a growing population of people resistant to taking prescription drugs. In part, these notions have contributed to a movement toward homeopathic remedies.

Over the counter anti-viral products currently available make claims to either provide relief of symptoms of an outbreak, to shorten healing time, or both. The over the counter product currently with the largest market share, is an ointment/cream that calls attention to the symptoms expressed on the face because of the product's highly visible nature—in spite of efforts to be more discreet by creating other product lines that combine its core product with skin-colored bandages to camouflage the cream. Because an HSV outbreak is not life threatening, is episodic rather than constant, but is highly visible on the face, there are psychological and social effects for those who suffer cold sores. Having the virus expressed as sores on the face is an embarrassment, a cause for self-consciousness, affects social interactions and life events, and is always a threat. Drawing more attention to the expressed symptoms is an unfortunate consequence when the product that is the most accessible and has the most positive results, if meager at best, is the best current over the counter solution.

The psycho-emotional aspect to HSV expressed as cold sores is a difficult aspect to document with data. Most references address psycho-emotional effects of HSV expressed in the genitals because of its stigma and the effects on sexual relations/health. Having a cold sore is not just uncomfortable and painful; it is embarrassing and can affect daily life, events, and interactions with others. The most expressive part of the body is visibly and grotesquely affected by a virus that a person has no control over and must live with forever. Human nature dictates a response to an attack on the body. A virus attacks the body internally. Although the most popular over the counter method of treatment widely accessible/available is an external defense addressing symptoms and boasts to only shorten duration of an outbreak, people with the virus continue to buy and use this product as their means for action. Taking action helps a person with the psychological aspect of suffering an episode.

Current prescription medicines and current over the counter medicines do not utilize allicin as the active ingredient in addressing HSV symptoms. It has been reported that introducing the presence of allicin in the body leads to increased antimicrobial activity by the body. The IUPAC name for allicin is S-Prop-2-en-1-yl prop-2-ene-1-sulfinothioate. Other chemical names for allicin include: 2-Propene-1-sulfinothioic acid S-2-propenyl ester; 3-[(Prop-2-ene-1-sulfinyl)sulfanyl]prop-1-ene; and S-Allyl prop-2-ene-1 -sul-finothioate. The molecular chemical structural formula of allicin is:

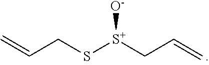

Allicin is a chiral chemical molecule, but occurs naturally as a racemate. When fresh garlic is chopped or crushed, the enzyme alliinase converts alliin into allicin, which is responsible for the aroma of fresh garlic. The allicin generated is unstable and quickly changes into a series of other sulfur-containing compounds such as diallyl disulfide.

### BRIEF SUMMARY OF THE INVENTION

Some embodiments of the present invention are a method of administering orally a therapeutically effective pharmaceutical formulation of allicin in a pharmaceutical dosage form to a patient with a herpes simplex viral infection (HSV-1 and/or HSV-2 infection), the therapeutically effective pharmaceutical formulation of allicin for increasing a human immune system activity in the patient during a reactivation of a herpes simplex virus causing a prodromal phase of the herpes simplex virus outbreak in the patient, the increasing of the human immune system activity in the patient for suppressing a herpes simplex viral symptom in the patient, the method comprising the steps of: administering orally a first therapeutically effective pharmaceutical formulation of the allicin with a dose of the allicin in an amount of between about 7,200 micrograms to about 12,000 micrograms in the pharmaceutical dosage form during onset of the prodromal phase of the herpes simplex virus outbreak in the patient; administering orally a second pharmaceutical formulation of the allicin with the dose of the allicin in the amount of between about 3000 micrograms to about 4800 micrograms in the pharmaceutical dosage form during the prodromal phase of the herpes simplex virus outbreak in the patient; administering orally a third pharmaceutical formulation of the allicin with the dose of the allicin in the amount of between about 3000 micrograms to about 4800 micrograms in the pharmaceutical dosage form during the prodromal phase of the herpes simplex virus outbreak in the patient; administering orally a fourth pharmaceutical formulation of the allicin with the dose of the allicin in the amount of between about 3000 micrograms to about 4800 micrograms in the pharmaceutical dosage form during the prodromal phase of the outbreak in the patient; administering orally a fifth oral dose in the amount of between about 3000 micrograms to about 4800 micrograms in the pharmaceutical dosage form during the prodromal phase of the herpes simplex virus outbreak in the patient; administering orally a sixth pharmaceutical formulation of the allicin with the dose of the allicin in the amount of between about 3000 micrograms to about 4800 micrograms in the pharmaceutical dosage form during the prodromal phase of the herpes simplex virus outbreak in the patient; administering orally a seventh pharmaceutical formulation of the allicin with the dose of the allicin in the amount of between about 3000 micrograms to about 4800 micrograms in the pharmaceutical dosage form during the prodromal phase of the herpes simplex virus outbreak in the patient; administering orally an eighth pharmaceutical formulation of the allicin with the dose of the allicin in the amount of between about 3000 micrograms to about 4800 micrograms in the pharmaceutical dosage form during the prodromal phase of the herpes simplex virus outbreak in the patient; administering orally a ninth pharmaceutical formulation of the allicin with the dose of the allicin in the amount of between about 3000 micrograms to about 4800 micrograms in the pharmaceutical dosage form during the prodromal phase of the herpes simplex virus outbreak in the patient; administering orally a tenth pharmaceutical formulation of the allicin with the dose of the allicin in the amount of between about 3000 micrograms to about 4800 micrograms in the pharmaceutical dosage form during the prodromal phase of the herpes simplex virus outbreak in the patient; and suppressing the herpes simplex virus symptom in the patient during the prodromal phase of the herpes simplex virus outbreak in the patient, and wherein the administering orally of the therapeutically effective pharmaceutical formulation of allicin in the pharmaceutical dosage form to the patient occurs over a time interval selected from the group consisting of hourly, daily, and a combination thereof.

Some embodiments of the present invention are a method of administering orally a therapeutically effective pharmaceutical formulation of allicin in a pharmaceutical dosage form to a patient with a herpes simplex viral infection, the therapeutically effective pharmaceutical formulation of allicin for increasing a human immune system activity in the patient during a reactivation of a herpes simplex virus causing a prodromal phase of the herpes simplex virus outbreak in the patient, the increasing of the human immune system activity in the patient for suppressing a herpes simplex viral symptom in the patient, the method comprising the steps of: administering orally a first therapeutically effective pharmaceutical formulation of the allicin with a dose of the allicin in an amount of between about 7,200 micrograms to about 12,000 micrograms in the pharmaceutical dosage form during onset of the prodromal phase of the herpes simplex virus outbreak in the patient; and subsequently administering orally at selected time intervals an additional nine doses of the pharmaceutical formulation of the allicin with the dose of the allicin in the amount of between about 3000 micrograms to about 4800 micrograms in the pharmaceutical dosage form during the prodromal phase of the herpes simplex virus outbreak in the patient, wherein the reactivation of the herpes simplex virus follows a period of latency in the patient, wherein the herpes simplex virus resides in the trigeminal ganglion in the patient, wherein the herpes simplex viral symptom in the patient is expressed on the face of the patient as a cold sore symptom of the herpes simplex virus, and wherein the initial dose of the allicin and subsequent doses administered to the patient at timed intervals enable a suppression of the herpes simplex viral symptom in the patient which is expressed on the face of the patient as a cold sore symptom of the herpes simplex virus. Also, some embodiments of the present invention are a method of administering orally a therapeutically effective pharmaceutical formulation of allicin in a pharmaceutical dosage form to a patient with a herpes simplex viral infection, the therapeutically effective pharmaceutical formulation of allicin for increasing a human immune system activity in the patient during a reactivation of a herpes simplex virus causing a prodromal phase of the herpes simplex virus outbreak in the patient, the increasing of the human immune system activity in the patient for suppressing a herpes simplex viral symptom in the patient, the method comprising the steps of: administering orally a first therapeutically effective pharmaceutical formulation of the allicin with a dose of the allicin in an amount of between about 7,200 micrograms to about 12,000 micrograms in the pharmaceutical dosage form during onset of the prodromal phase of the herpes simplex virus outbreak in the patient; and subsequently administering orally at selected time intervals an additional nine doses of the pharmaceutical formulation of the allicin with the dose of the allicin in the amount of between about 3000 micrograms to about 4800 micrograms in the pharmaceutical dosage form during the prodromal phase of the herpes simplex virus outbreak in the patient, wherein the reactivation of the herpes simplex virus follows a period of latency in the patient, wherein the herpes simplex virus resides in the sacral ganglion in the patient, wherein the herpes simplex viral symptom in the patient is expressed on a genital area of the patient as a genital sore symptom of the herpes simplex virus, and wherein the initial dose of the allicin and subsequent doses administered to the patient at timed intervals enable a suppression of the herpes simplex viral symptom in the patient which is expressed in the genital area of the patient as a sore or blister symptom of the herpes simplex virus.

In some embodiments of the present invention, the oral pharmaceutical formulation of the allicin with the dose of the allicin may be a capsule pharmaceutical dosage form. In some embodiments of the present invention, the oral pharmaceutical formulation of the allicin with the dose of the allicin may further comprise an additional ingredient selected from the group consisting of 10 mg lysine, a vanilla flavoring, a vanilla ingredient, a lemon verbena, and a combination thereof. In some embodiments of the present invention, the oral pharmaceutical formulation of the allicin with the dose of the allicin may be pharmaceutical dosage form selected from the group consisting of a tablet pharmaceutical dosage form, and a gelcap pharmaceutical dosage form. Some embodiments of the present invention, further comprise an additional ingredient selected from the group consisting of 10 mg lysine, a vanilla coating, a vanilla ingredient, a lemon verbena, and a combination thereof.

In some embodiments of the present invention is a method of administering orally a therapeutically effective pharmaceutical formulation of allicin in a pharmaceutical dosage form to a patient with a herpes simplex viral infection, the therapeutically effective pharmaceutical formulation of allicin for increasing a human immune system activity in the patient during a reactivation of a herpes simplex virus causing a prodromal phase of the herpes simplex virus outbreak in the patient, the increasing of the human immune system activity in the patient for suppressing a herpes simplex viral symptom in the patient, the method comprising the steps of: administering orally a first therapeutically effective pharmaceutical formulation of the allicin with a dose of the allicin in an amount of between about 7,200 micrograms to about 12,000 micrograms during onset of the prodromal phase of the herpes simplex virus outbreak in the patient; administering orally a second pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient; administering orally a third pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient; administering orally a fourth pharmaceutical formulation of the allicin with the dose of the allicin in the amount of between about 3,000 micrograms to about 4,800 micrograms in the pharmaceutical dosage form during the prodromal phase of the outbreak in the patient; administering orally a fifth oral dose in the amount of between about 3,000 micrograms to about 4,800 micrograms in the pharmaceutical dosage form during the prodromal phase of the herpes simplex virus outbreak in the patient; administering orally a sixth pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient; administering orally a seventh pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient; administering orally an eighth pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient; administering orally a ninth pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient; administering orally a tenth pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient; and suppressing the herpes simplex virus symptom in the patient during the prodromal phase of the herpes simplex virus outbreak in the patient.

In some embodiments of the invention the therapeutically effective pharmaceutical formulation of allicin in the pharmaceutical dosage form to the patient is administered over a time interval selected from the group consisting of hourly, daily, and a combination thereof. In some embodiments of the invention the oral pharmaceutical formulation of the aliicin is administered in the pharmaceutical dosage form selected from the group consisting of a capsule pharmaceutical dosage form, a tablet pharmaceutical dosage form, and a gelcap pharmaceutical dosage form, and a combination thereof. Some embodiments of the invention relate to methods of administering orally a therapeutically effective pharmaceutical formulation of between about 3000 micrograms to about 12,000 micrograms of allicin in a pharmaceutical dosage form to a patient with a herpes simplex viral infection (HSV-1 and/or HSV-2 infection), the therapeutically effective pharmaceutical formulation of allicin for increasing a human immune system activity in the patient during a reactivation of a herpes simplex virus causing a prodromal phase of the herpes simplex virus outbreak in the patient, the increasing of the human immune system activity in the patient for suppressing a herpes simplex viral symptom in the patient. In some embodiments, allicin doses are administered hourly or daily for a total of ten dose administrations or until a herpes simplex viral symptom is suppressed. Procedures for making capsules, tablets and gelcaps are well known in the art of pharmaceutical formulation and pharmaceutical dosage forms.

### BRIEF DESCRIPTION OF DRAWINGS

FIG. 1 is a flowchart illustrating a method for some embodiments of the present invention comprising administering a first oral dose of allicin to a patient during onset of a prodromal phase of a herpes simple outbreak in the patient, and subsequently administering additional oral doses of the allicin to the patient during the prodromal phase of the herpes simple outbreak in the patient until prodromal sensations pass in the patient.

FIG. 2 is a flowchart in accordance with FIG. 1 that illustrates a method for some embodiments of the present invention comprising administering nine oral doses of allicin to a patient following an administration of a first oral dose of the allicin to the patient.

FIG. 3 is a flowchart in accordance with FIG. 1 that illustrates a method for some embodiments of the present invention comprising administering nine oral doses of allicin to a patient following an administration of a first oral dose of the allicin to the patient, wherein the doses of the allicin are administered hourly.

FIG. 4 is a flowchart in accordance with FIG. 1 that illustrates a method for some embodiments of the present invention comprising administering nine oral doses of allicin to a patient following an administration of a first oral dose of the allicin to the patient, wherein the doses of the allicin are administered daily.

FIG. 5 illustrates capsules with a specific range of allicin dosages for some embodiments of the present invention to be used for administering ten oral capsule doses of allicin.

FIG. 6 illustrates tablets with a specific range of allicin dosages for some embodiments of the present invention to be used for administering ten oral capsule doses of allicin.

FIG. 7 illustrates gelcaps with a specific range of allicin dosages to be used for administering for ten oral gelcap doses of allicin.

FIG. 8 illustrates capsules with a specific range of allicin dosages to be used for administering ten oral capsule doses of allicin and capsules with a specific dose amount of lysine to be used for administering ten oral capsule doses of lysine.

FIG. 9 illustrates tablets with a specific range of allicin dosages to be used for administering ten oral tablet doses of allicin and tablets with a specific dose amount of lysine to be used for administering ten oral tablet doses of lysine.

FIG. 10 illustrates gelcaps with a specific range of allicin dosages to be used for administering ten oral gelcap doses of allicin and gelcaps with a specific dose amount of lysine to be used for administering ten oral doses of lysine.

FIG. 11 illustrates capsules with a specific range of allicin dosages to be used for administering ten oral capsule doses of allicin and capsules with a specific dose amount of lysine to be used for administering ten oral doses of lysine and a vanilla coating on each of the capsules.

FIG. 12 illustrates tablets with a specific range of allicin dosages to be used for administering ten oral tablet doses of allicin and tablets with a specific dose amount of lysine to be used for administering ten oral doses of lysine and a vanilla coating on each of the tablets.

FIG. 13 illustrates gelcaps with a specific range of allicin dosages to be used for administering ten oral tablet doses of allicin and gelcaps with a specific dose amount of lysine to be used for administering ten oral doses of lysine and a vanilla coating on each of the gelcaps.

FIG. 14 illustrates capsules a specific range of allicin dosages to be used for administering ten oral doses of allicin and capsules with a specific dose amount of lysine to be used for administering ten oral doses of lysine and a lemon verbena coating on each of the capsules.

FIG. 15 illustrates tablets with a specific range of allicin dosages to be used for administering ten oral doses of allicin and tablets with a specific dose amount of lysine to be used for administering ten oral doses of lysine and a lemon verbena coating on each of the capsules.

FIG. 16 illustrates gelcaps with a specific range of allicin dosages to be used for administering ten oral doses of allicin and gelcaps with a specific dose amount of lysine to be used for administering ten oral doses of lysine and with a lemon verbena coating on each of the gelcaps.

### DETAILED DESCRIPTION OF INVENTION

The inventor has invented in improved method of using allicin to supplement a human immune system during the onset of the prodromal phase of an Herpes Simplex Virus (HSV)-1 and/or HSV-2) episode (an episode has the same meaning as a outbreak), following the reactivation of HSV residing in the trigeminal ganglion, with a critical amount of allicin, thereby enabling the immune system to receive the amount of allicin needed at the time it is needed to effectively suppress the appearance of HSV symptoms expressed on the face (Herpes Labialis, commonly “cold sores”), comprised of a critical first dosage of no less than 7200 mcg (micrograms) and up to 12,000 mcg of allicin, ingested during the onset of the prodromal phase, followed by subsequently ingested critical dosages of no less than 3000 mcg and up to 4800 mcg in each of the said subsequent dosages until prodromal symptoms are no longer sensed. Note that mcg means micrograms Oral administering of allicin increases antimicrobial activity against herpes simplex virus (HSV-1 and/or HSV-2) in the body, thereby aiding the immune system. Methods of allicin administration, allicin pharmaceutical formulations, and allicin pharmaceutical dosage formulations disclosed in Ott, U.S. Pat. No. 8,222,299; and Chakraborty, U.S. Pat. No. 9,192,651 are herein incorporated by reference in their entirety.

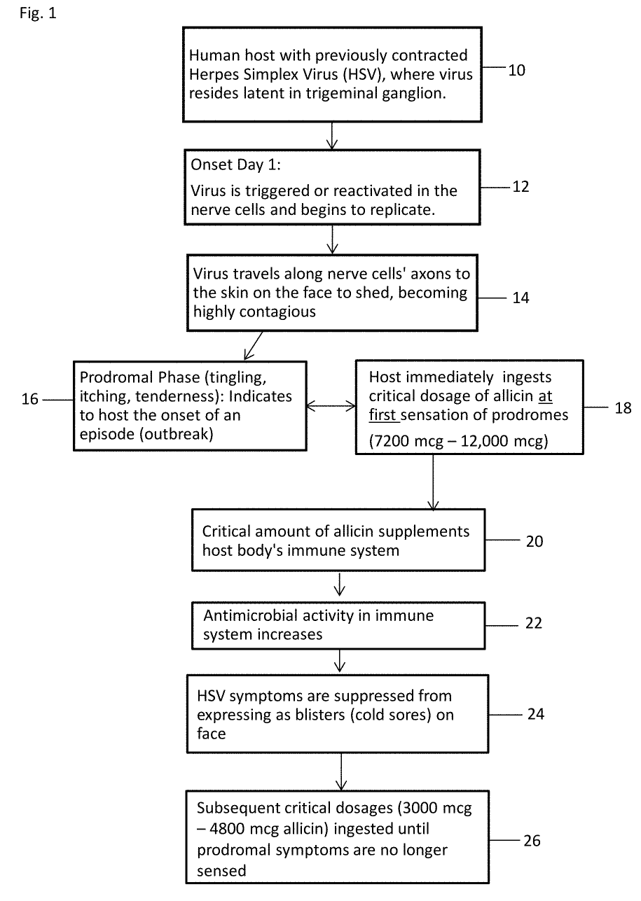

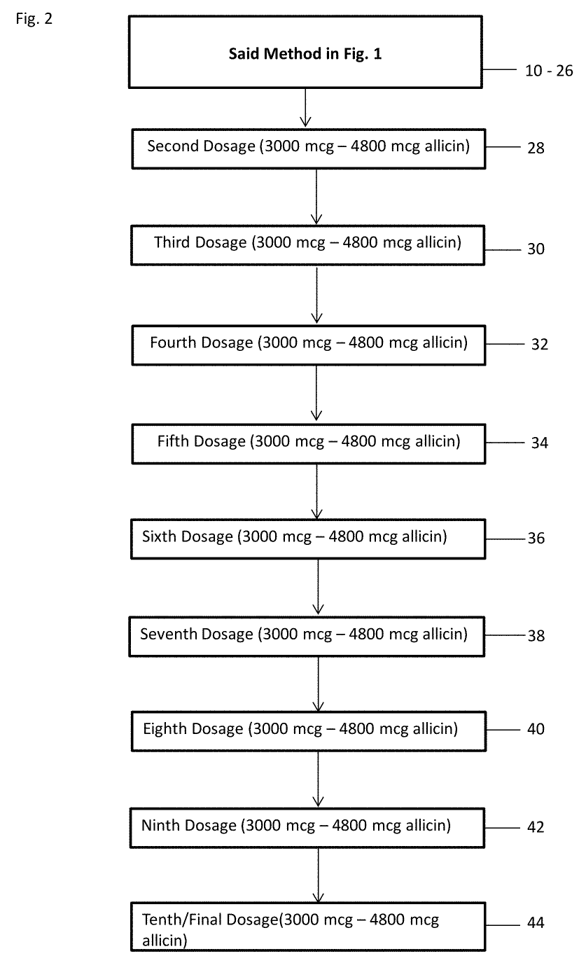

Referring now to the invention in more detail, shown in FIG. 1 flowchart illustrates a human host with previously contracted Herpes Simplex Virus (HSV), wherein said virus resides latent in the trigeminal ganglion 10. Said virus is triggered or reactivated into replication and begins the onset of an HSV episode 12. Said virus travels along the host's nerve cell's axons to shed on the face (often the lips), becoming highly contagious 14 and is accompanied by sensations of tingling, itching, tenderness, which is the host's first indication of the prodromal phase of said virus and simultaneous need for initial critical dosage of allicin 16.

Host immediately ingests the initial critical dosage of no less than 7200 mcg of allicin and up to 12,000 mcg of allicin 18. Said initial critical dosage 18, leads to the host body's response, where a critical amount of allicin is introduced to the body at the critical time in the virus's progression 20, antimicrobial activity against herpes simplex virus (HSV-1 and/or HSV-2) is boosted 22 to supplement the body's immune system when the herpes simplex virus has exposed itself, but has not yet replicated enough to take an indefensible hold and express itself through symptoms, thereby suppressing the appearance of symptoms expressed as cold sores on the face 24. Subsequent critical dosages 26 are taken, where said subsequent critical dosages are comprised of no less than 3000 mcg of allicin and up to 4800 mcg of allicin, and ingested until prodromal sensations are no longer sensed.

Shown in FIG. 2 is a flowchart to illustrate said method of use of allicin in reference numbers 10-26 of FIG. 1, wherein said initial critical dosage is followed by a subsequent second critical dosage of no less than 3000 mcg and up to 4800 mcg of allicin 28. Said second dosage 28 is followed by a third critical dosage of no less than 3000 mcg and up to 4800 mcg of allicin 30. Said third critical dosage 30 is followed by a fourth critical dosage of no less than 3000 mcg and up to 4800 mcg of allicin 32. Said fourth critical dosage 32 is followed by a fifth critical dosage of no less than 3000 mcg and up to 4800 mcg of allicin 34. Said fifth critical dosage 34 is followed by a sixth critical dosage of no less than 3000 mcg and up to 4800 mcg of allicin 36. Said sixth critical dosage 36 is followed by a seventh critical dosage of no less than 3000 mcg and up to 4800 mcg of allicin 38. Said seventh critical dosage 38 is followed by an eighth critical dosage of no less than 3000 mcg and up to 4800 mcg of allicin 40. Said eighth critical dosage 40 is followed by a ninth critical dosage of no less than 3000 mcg and up to 4800 mcg of allicin 42. Said ninth critical dosage is followed by a tenth critical dosage of no less than 3000 mcg and up to 4800 mcg of allicin 44.

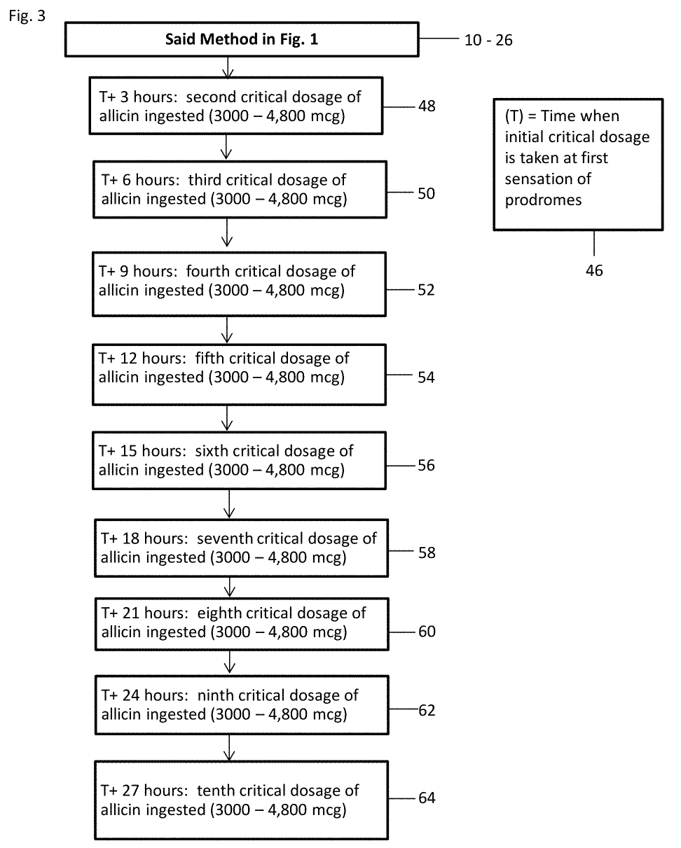

Shown in FIG. 3 is a flowchart to illustrate said method of use of allicin in reference numbers 10-26 of FIG. 1, wherein said subsequent critical dosages are nine in number and ingested every 3 hours following said initial critical dosage 18. For illustrative purposes, the host's first sensation of said prodromal phase 16 and time initial critical dosage is ingested 18 will be represented by T 46. Said initial critical ingested dosage 18 is followed by a second critical dosage of no less than 3000 mcg and up to 4800 mcg of allicin ingested at T+3 hours 48. Said second dosage 48 is followed by a third critical dosage of no less than 3000 mcg and up to 4800 mcg of allicin ingested at T+6 hours 50. Said third critical dosage 50 is followed by a fourth critical dosage of no less than 3000 mcg and up to 4800 mcg of allicin ingested at T+9 hours 52. Said fourth critical dosage 52 is followed by a fifth critical dosage of no less than 3000 mcg and up to 4800 mcg of allicin ingested at T+12 hours 54. Said fifth critical dosage 54 is followed by a sixth critical dosage of no less than 3000 mcg and up to 4800 mcg of allicin ingested at T+15 hours 56. Said sixth critical dosage 56 is followed by a seventh critical dosage of no less than 3000 mcg and up to 4800 mcg of allicin ingested at T+18 hours 58. Said seventh critical dosage 58 is followed by an eighth critical dosage of no less than 3000 mcg and up to 4800 mcg of allicin ingested at T+21 hours 60. Said eighth critical dosage 60 is followed by a ninth critical dosage of no less than 3000 mcg and up to 4800 mcg of allicin ingested at T+24 hours 62. Said ninth critical dosage 62 is followed by a tenth critical dosage of no less than 3000 mcg and up to 4800 mcg of allicin ingested at T+27 hours 64.

Shown in FIG. 4 is a flowchart to illustrate said method of use of allicin in reference numbers 10-26 of FIG. 1, wherein said subsequent critical dosages 26 are nine in number, ingested every three hours following the initial critical dose 18 on Day One 68, and three times per day on Day Two 76 and Day Three 84, in morning, afternoon, and evening. For illustrative purposes, the host's first sensation of said prodromal phase 16 and time initial critical dosage is ingested 18 will be represented by X 66. Said initial critical ingested dosage 18 is followed by a second critical dosage of no less than 3000 mcg and up to 4800 mcg of allicin ingested at X+3 hours 70. Said second dosage 70 is followed by a third critical dosage of no less than 3000 mcg and up to 4800 mcg of allicin ingested at X+6 hours 72. Said third critical dosage 72 is followed by a fourth critical dosage of no less than 3000 mcg and up to 4800 mcg of allicin ingested at X+9 hours 74. On Day Two 76, a fifth critical dosage of no less than 3000 mcg and up to 4800 mcg of allicin ingested in the morning 78. Said fifth critical dosage 78 is followed by a sixth critical dosage of no less than 3000 mcg and up to 4800 mcg of allicin ingested in the afternoon 80. Said sixth critical dosage 80 is followed by a seventh critical dosage of no less than 3000 mcg and up to 4800 mcg of allicin ingested in the evening 82. On Day Three 84, an eighth critical dosage of no less than 3000 mcg and up to 4800 mcg of allicin ingested in the morning 86. Said eighth critical dosage 86 is followed by a ninth critical dosage of no less than 3000 mcg and up to 4800 mcg of allicin ingested in the afternoon 88. Said ninth critical dosage 88 is followed by a tenth critical dosage of no less than 3000 mcg and up to 4800 mcg of allicin ingested in the evening 90.

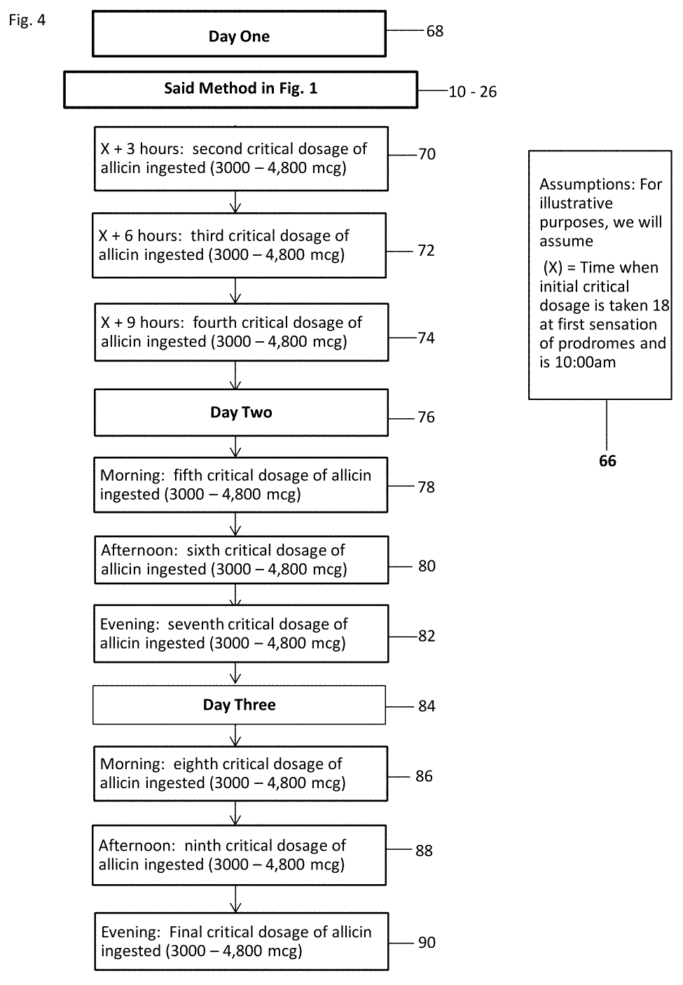

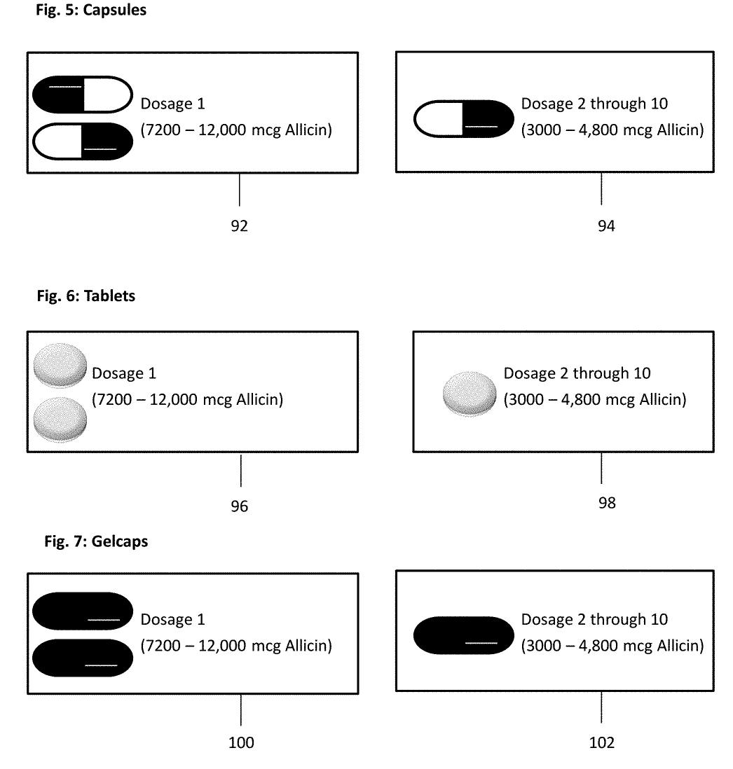

FIG. 5 illustrates said initial critical dosage of allicin 18 when said dosage is in capsule form 92 and said subsequent critical dosages of allicin 26 when said dosages are in capsule form 94.

FIG. 6 illustrates said initial critical dosage of allicin 18 when said dosage is in tablet form 96 and said subsequent critical dosages of allicin 26 when said dosages are in tablet form 98.

FIG. 7 illustrates said initial critical dosage of allicin 18 when said dosage is in gelcap form 100 and said subsequent critical dosages of allicin 26 when said dosages are in gelcap form 102.

FIG. 8 illustrates said initial critical dosage 18 when said dosage is comprised of no less than 7200 mcg and up to 12,000 mcg of allicin, 10 mg of Lysine, is in capsule form 104 and said subsequent critical dosages 26, when said dosages are comprised of no less than 3000 mcg and up to 4800 mcg of allicin, 10 mg of Lysine, and in capsule form 106.

FIG. 9 illustrates said initial critical dosage 18 when said dosage is comprised of no less than 7200 mcg and up to 12,000 mcg of allicin, 10 mg of Lysine, is in tablet form 108 and said subsequent critical dosages 26, when said dosages are comprised of no less than 3000 mcg and up to 4800 mcg of allicin, 10 mg of Lysine, and in tablet form 110.

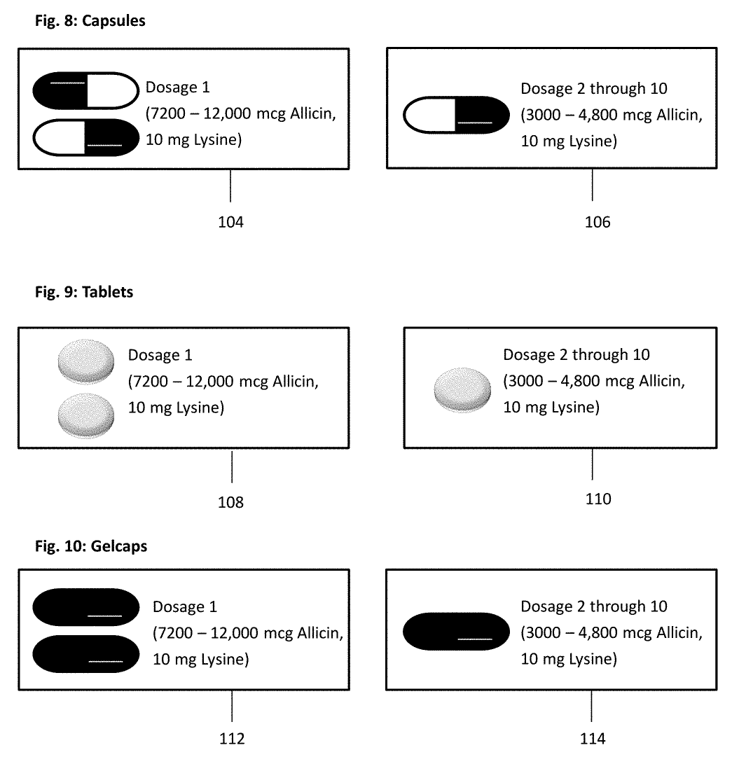

FIG. 10 illustrates said initial critical dosage 18 when said dosage is comprised of no less than 7200 mcg and up to 12,000 mcg of allicin, 10 mg of Lysine, is in gelcap form 112 and said subsequent critical dosages 26, when said dosages are comprised of no less than 3000 mcg and up to 4800 mcg of allicin, 10 mg of Lysine, and in gelcap form 114.

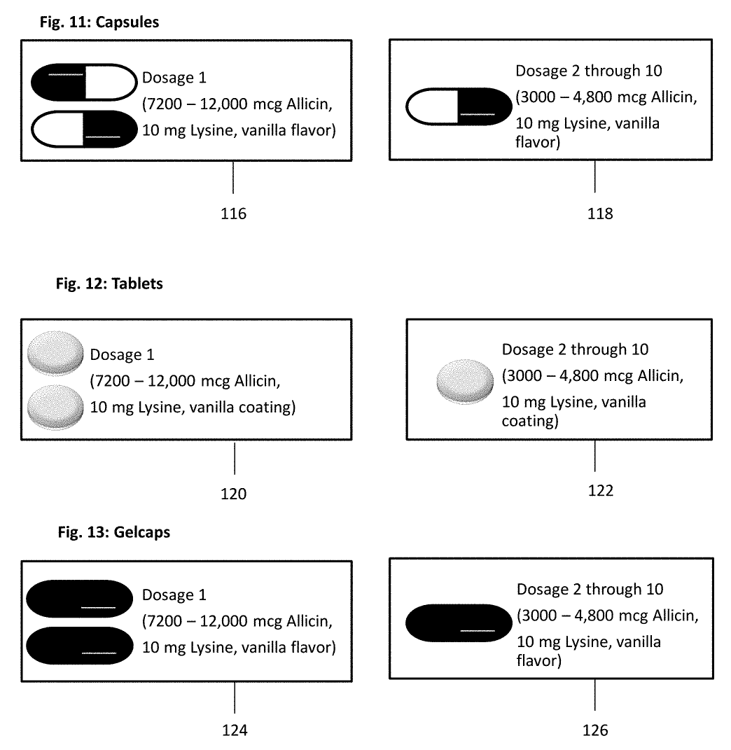

FIG. 11 illustrates said initial critical dosage 18 when said dosage is comprised of no less than 7200 mcg and up to 12,000 mcg of allicin, 10 mg of Lysine, vanilla flavor, is in capsule form 116 and said subsequent critical dosages 26, when said dosages are comprised of no less than 3000 mcg and up to 4800 mcg of allicin, 10 mg of Lysine, vanilla flavor, and in capsule form 118.

FIG. 12 illustrates said initial critical dosage 18 when said dosage is comprised of no less than 7200 mcg and up to 12,000 mcg of allicin, 10 mg of Lysine, vanilla coating, is in tablet form 120 and said subsequent critical dosages 26, when said dosages are comprised of no less than 3000 mcg and up to 4800 mcg of allicin, 10 mg of Lysine, vanilla coating, and in tablet form 122.

FIG. 13 illustrates said initial critical dosage 18 when said dosage is comprised of no less than 7200 mcg and up to 12,000 mcg of allicin, 10 mg of Lysine, vanilla flavor, is in gelcap form 124 and said subsequent critical dosages 26, when said dosages are comprised of no less than 3000 mcg and up to 4800 mcg of allicin, 10 mg of Lysine, vanilla flavor, and in gelcap form 126.

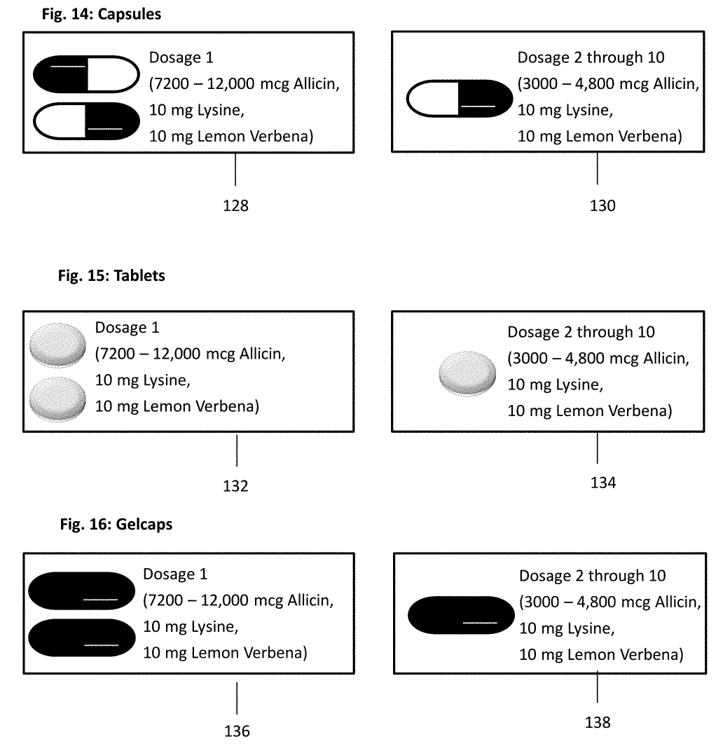

FIG. 14 illustrates said initial critical dosage 18 when said dosage is comprised of no less than 7200 mcg and up to 12,000 mcg of allicin, 10 mg of Lysine, 10 mg Lemon Verbena, is in capsule form 128 and said subsequent critical dosages 26, when said dosages are comprised of no less than 3000 mcg and up to 4800 mcg of allicin, 10 mg of Lysine, 10 mg Lemon Verbena and in capsule form 130.

FIG. 15 illustrates said initial critical dosage 18 when said dosage is comprised of no less than 7200 mcg and up to 12,000 mcg of allicin, 10 mg of Lysine, 10 mg Lemon Verbena, is in tablet form 132 and said subsequent critical dosages 26, when said dosages are comprised of no less than 3000 mcg and up to 4800 mcg of allicin, 10 mg of Lysine, 10 mg Lemon Verbena and in tablet form 134.

FIG. 16 illustrates said initial critical dosage 18 when said dosage is comprised of no less than 7200 mcg and up to 12,000 mcg of allicin, 10 mg of Lysine, 10 mg Lemon Verbena, is in gelcap form 136 and said subsequent critical dosages 26, when said dosages are comprised of no less than 3000 mcg and up to 4800 mcg of allicin, 10 mg of Lysine, 10 mg Lemon Verbena and in gelcap form 138.

Some embodiments of the present invention are a method of administering orally a therapeutically effective pharmaceutical formulation of allicin in a pharmaceutical dosage form to a patient with a herpes simplex viral infection (HSV-1 and/or HSV-2 infection), the therapeutically effective pharmaceutical formulation of allicin for increasing a human immune system activity in the patient during a reactivation of a herpes simplex virus causing a prodromal phase of the herpes simplex virus outbreak in the patient, the increasing of the human immune system activity in the patient for suppressing a herpes simplex viral symptom in the patient, the method comprising the steps of: administering orally a first therapeutically effective pharmaceutical formulation of the allicin with a dose of the allicin in an amount of between about 7,200 micrograms to about 12,000 micrograms in the pharmaceutical dosage form during onset of the prodromal phase of the herpes simplex virus outbreak in the patient; administering orally a second pharmaceutical formulation of the allicin with the dose of the allicin in the amount of between about 3000 micrograms to about 4800 micrograms in the pharmaceutical dosage form during the prodromal phase of the herpes simplex virus outbreak in the patient; administering orally a third pharmaceutical formulation of the allicin with the dose of the allicin in the amount of between about 3000 micrograms to about 4800 micrograms in the pharmaceutical dosage form during the prodromal phase of the herpes simplex virus outbreak in the patient; administering orally a fourth pharmaceutical formulation of the allicin with the dose of the allicin in the amount of between about 3000 micrograms to about 4800 micrograms in the pharmaceutical dosage form during the prodromal phase of the outbreak in the patient; administering orally a fifth oral dose in the amount of between about 3000 micrograms to about 4800 micrograms in the pharmaceutical dosage form during the prodromal phase of the herpes simplex virus outbreak in the patient; administering orally a sixth pharmaceutical formulation of the allicin with the dose of the allicin in the amount of between about 3000 micrograms to about 4800 micrograms in the pharmaceutical dosage form during the prodromal phase of the herpes simplex virus outbreak in the patient; administering orally a seventh pharmaceutical formulation of the allicin with the dose of the allicin in the amount of between about 3000 micrograms to about 4800 micrograms in the pharmaceutical dosage form during the prodromal phase of the herpes simplex virus outbreak in the patient; administering orally an eighth pharmaceutical formulation of the allicin with the dose of the allicin in the amount of between about 3000 micrograms to about 4800 micrograms in the pharmaceutical dosage form during the prodromal phase of the herpes simplex virus outbreak in the patient; administering orally a ninth pharmaceutical formulation of the allicin with the dose of the allicin in the amount of between about 3000 micrograms to about 4800 micrograms in the pharmaceutical dosage form during the prodromal phase of the herpes simplex virus outbreak in the patient; administering orally a tenth pharmaceutical formulation of the allicin with the dose of the allicin in the amount of between about 3000 micrograms to about 4800 micrograms in the pharmaceutical dosage form during the prodromal phase of the herpes simplex virus outbreak in the patient; and suppressing the herpes simplex virus symptom in the patient during the prodromal phase of the herpes simplex virus outbreak in the patient, and wherein the administering orally of the therapeutically effective pharmaceutical formulation of allicin in the pharmaceutical dosage form to the patient occurs over a time interval selected from the group consisting of hourly, daily, and a combination thereof.

In addition, some embodiments of the present invention are a method of administering orally a therapeutically effective pharmaceutical formulation of allicin in a pharmaceutical dosage form to a patient with a herpes simplex viral infection (HSV-1 and/or HSV-2 infection), the therapeutically effective pharmaceutical formulation of allicin for increasing a human immune system activity in the patient during a reactivation of a herpes simplex virus causing a prodromal phase of the herpes simplex virus outbreak in the patient, the increasing of the human immune system activity in the patient for suppressing a herpes simplex viral symptom in the patient, the method comprising the steps of: administering orally a first dose of a therapeutically effective pharmaceutical formulation of the allicin with a dosage amount of the allicin in the dosage amount selected from the group consisting of between about 100 micrograms to about 200,000 micrograms, in an amount of between about 300 micrograms to about 150,000 micrograms, in an amount of between about 1,000 micrograms to about 100,000 micrograms, in an amount of between about 1,000 micrograms to about 50,000 micrograms, in an amount of between about 1,000 micrograms to about 25,000 micrograms, in an amount of between about 2,000 micrograms to about 20,000 micrograms, in an amount of between about 3,000 micrograms to about 20,000 micrograms, in an amount of between about 4,000 micrograms to about 20,000 micrograms, in an amount of between about 5,000 micrograms to about 17,000 micrograms, in an amount of between about 5,000 micrograms to about 15,000 micrograms, in an amount of between about 6,000 micrograms to about 13,000 micrograms, in an amount of between about 6,000 micrograms to about 12,000 micrograms, in an amount of between about 7,000 micrograms to about 12,000 micrograms, in an amount of between about 7,200 micrograms to about 12,000 micrograms, in an amount of between about 8,000 micrograms to about 11,000 micrograms, and in an amount of between about 8,000 micrograms to about 10,000 micrograms in the pharmaceutical dosage form during onset of the prodromal phase of the herpes simplex virus outbreak in the patient; and administering orally an additional number of doses of a pharmaceutical formulation of the allicin with a dosage amount of the allicin in the dosage amount selected from the group consisting of between about 100 micrograms to about 200,000 micrograms, in an amount of between about 300 micrograms to about 150,000 micrograms, in an amount of between about 1,000 micrograms to about 100,000 micrograms, in an amount of between about 1,000 micrograms to about 50,000 micrograms, in an amount of between about 1,000 micrograms to about 25,000 micrograms, in an amount of between about 2,000 micrograms to about 20,000 micrograms, in an amount of between about 3,000 micrograms to about 20,000 micrograms, in an amount of between about 4,000 micrograms to about 20,000 micrograms, in an amount of between about 5,000 micrograms to about 17,000 micrograms, in an amount of between about 5,000 micrograms to about 15,000 micrograms, in an amount of between about 6,000 micrograms to about 13,000 micrograms, in an amount of between about 6,000 micrograms to about 12,000 micrograms, in an amount of between about 7,000 micrograms to about 12,000 micrograms, in an amount of between about 7,200 micrograms to about 12,000 micrograms, in an amount of between about 8,000 micrograms to about 11,000 micrograms, in an amount of between about 8,000 micrograms to about 10,000 micrograms, in the amount of between about 3000 micrograms to about 4800 micrograms, and any comination thereof in any known or herein named pharmaceutical dosage form preferably during the prodromal phase of the herpes simplex virus outbreak in the patient, wherein the additional number of doses of a pharmaceutical formulation of the allicin is selected from the group consisting of no additional dose of a pharmaceutical formulation of the allicin, one additional dose of a pharmaceutical formulation of the allicin, two additional doses of a pharmaceutical formulation of the allicin, three additional doses of a pharmaceutical formulation of the allicin, four additional doses of a pharmaceutical formulation of the allicin, five additional doses of a pharmaceutical formulation of the allicin, six additional doses of a pharmaceutical formulation of the allicin, seven additional doses of a pharmaceutical formulation of the allicin, eight additional doses of a pharmaceutical formulation of the allicin, nine additional doses of a pharmaceutical formulation of the allicin, ten additional doses of a pharmaceutical formulation of the allicin, eleven additional doses of a pharmaceutical formulation of the allicin, twelve additional doses of a pharmaceutical formulation of the allicin, thirteen additional doses of a pharmaceutical formulation of the allicin, fourteen additional doses of a pharmaceutical formulation of the allicin, fifteen additional doses of a pharmaceutical formulation of the allicin, sixteen additional doses of a pharmaceutical formulation of the allicin, seventeen additional doses of a pharmaceutical formulation of the allicin, eighteen additional doses of a pharmaceutical formulation of the allicin, nineteen additional doses of a pharmaceutical formulation of the allicin, twenty additional doses of a pharmaceutical formulation of the allicin, twenty-one additional doses of a pharmaceutical formulation of the allicin, twenty-two additional doses of a pharmaceutical formulation of the allicin, twenty-three additional doses of a pharmaceutical formulation of the allicin, twenty-four additional doses of a pharmaceutical formulation of the allicin, twenty-five additional doses of a pharmaceutical formulation of the allicin, twenty-six additional doses of a pharmaceutical formulation of the allicin, twenty-seven additional doses of a pharmaceutical formulation of the allicin, twenty eight additional doses of a pharmaceutical formulation of the allicin, twenty-nine additional doses of a pharmaceutical formulation of the allicin, thirty additional doses of a pharmaceutical formulation of the allicin, and thirty-one additional doses of a pharmaceutical formulation of the allicin, wherein the additional doses of a pharmaceutical formulation of the allicin are administered at a timed allicin dosing interval selected from the group consisting of every hour, every two hours, every three hours, every four hours, every six hours, every twelve hours, daily, every two days, every three days, every week, every two weeks, monthly, and any combination thereof.

Instead of the method of orally administering the dose(s) of a pharmaceutical formulation of the allicin, some embodiments of the present invention area method of administering the dose(s) of a pharmaceutical formulation of the allicin by an administration route selected from the group consisting of nasal, inhalation, parenteral, intravenous, subcutaneous, intramuscular, topical cream, topical ointment, rectal suppository, vaginal, uretral, rectal, transdermal patch, sustained release tablet, sustained release capsule, subdermal, subcutaneous slow release implant, sublingual, and any combination thereof.

Some embodiments of the present invention are a method of administering orally a therapeutically effective pharmaceutical formulation of allicin in a pharmaceutical dosage form to a patient with a herpes simplex viral infection, the therapeutically effective pharmaceutical formulation of allicin for increasing a human immune system activity in the patient during a reactivation of a herpes simplex virus causing a prodromal phase of the herpes simplex virus outbreak in the patient, the increasing of the human immune system activity in the patient for suppressing a herpes simplex viral symptom in the patient, the method comprising the steps of: administering orally a first therapeutically effective pharmaceutical formulation of the allicin with a dose of the allicin in an amount of between about 3,000 micrograms to about 15,000 micrograms in the pharmaceutical dosage form during onset of the prodromal phase of the herpes simplex virus outbreak in the patient; and subsequently administering orally at selected time intervals an additional nine doses of the pharmaceutical formulation of the allicin with the dose of the allicin in the amount of between about 1000 micrograms to about 6000 micrograms in the pharmaceutical dosage form during the prodromal phase of the herpes simplex virus outbreak in the patient, wherein the reactivation of the herpes simplex virus follows a period of latency in the patient, wherein the herpes simplex virus resides in the trigeminal ganglion in the patient, wherein the herpes simplex viral symptom in the patient is expressed on the face of the patient as a cold sore symptom of the herpes simplex virus, and wherein the initial dose of the allicin and subsequent doses administered to the patient at timed allicin dosing interval which enables a suppression of the herpes simplex viral symptom in the patient which is expressed on the face of the patient as a cold sore symptom of the herpes simplex virus. Also, some embodiments of the present invention are a method of administering orally a therapeutically effective pharmaceutical formulation of allicin in a pharmaceutical dosage form to a patient with a herpes simplex viral infection, the therapeutically effective pharmaceutical formulation of allicin for increasing a human immune system activity in the patient during a reactivation of a herpes simplex virus causing a prodromal phase of the herpes simplex virus outbreak in the patient, the increasing of the human immune system activity in the patient for suppressing a herpes simplex viral symptom in the patient, the method comprising the steps of: administering orally a first therapeutically effective pharmaceutical formulation of the allicin with a dose of the allicin in an amount of between about 3,000 micrograms to about 25,000 micrograms in the pharmaceutical dosage form during onset of the prodromal phase of the herpes simplex virus outbreak in the patient; and subsequently administering orally at selected time intervals an additional nine doses of the pharmaceutical formulation of the allicin with the dose of the allicin in the amount of between about 1,000 micrograms to about 10,000 micrograms in the pharmaceutical dosage form during the prodromal phase of the herpes simplex virus outbreak in the patient, wherein the reactivation of the herpes simplex virus follows a period of latency in the patient, wherein the herpes simplex virus resides in the sacral ganglion in the patient, wherein the herpes simplex viral symptom in the patient is expressed on a genital area of the patient as a genital sore symptom of the herpes simplex virus, and wherein the initial dose of the allicin and subsequent doses administered to the patient at timed intervals enable a suppression of the herpes simplex viral symptom in the patient which is expressed in the genital area of the patient as a sore or blister symptom of the herpes simplex virus.

In some embodiments of the present invention, the oral pharmaceutical formulation of the allicin with the dose of the allicin may be a capsule pharmaceutical dosage form. In some embodiments of the present invention, the oral pharmaceutical formulation of the allicin with the dose of the allicin may further comprise an additional ingredient selected from the group consisting of 10 mg lysine, a vanilla flavoring, a vanilla ingredient, a lemon verbena, and a combination thereof. In some embodiments of the present invention, the oral pharmaceutical formulation of the allicin with the dose of the allicin may be pharmaceutical dosage form selected from the group consisting of a tablet pharmaceutical dosage form, and a gelcap pharmaceutical dosage form. Some embodiments of the present invention, further comprise an additional ingredient selected from the group consisting of 10 mg lysine, a vanilla coating, a vanilla ingredient, a lemon verbena, and a combination thereof. The additional ingredient amount of lysine may be selected from the group consisting of 1 mg, 2 mg, 3 mg, 4, mg, 5 mg, 10 mg, 15 mg, 20 mg, 30 mg, 50 mg, and any combination thereof. Any flavor, flavored coating, antioxidant, scent, or pharmaceutically acceptable excipient may be included in the allicin dosage form or allicin pharmaceutical formulation.

In some embodiments of the present invention is a method of administering orally a therapeutically effective pharmaceutical formulation of allicin in a pharmaceutical dosage form to a patient with a herpes simplex viral infection, the therapeutically effective pharmaceutical formulation of allicin for increasing a human immune system activity in the patient during a reactivation of a herpes simplex virus causing a prodromal phase of the herpes simplex virus outbreak in the patient, the increasing of the human immune system activity in the patient for suppressing a herpes simplex viral symptom in the patient, the method comprising the steps of: administering orally a first therapeutically effective pharmaceutical formulation of the allicin with a dose of the allicin in an amount of between about 7,200 micrograms to about 12,000 micrograms during onset of the prodromal phase of the herpes simplex virus outbreak in the patient; administering orally a second pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient; administering orally a third pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient; administering orally a fourth pharmaceutical formulation of the allicin with the dose of the allicin in the amount of between about 3,000 micrograms to about 4,800 micrograms in the pharmaceutical dosage form during the prodromal phase of the outbreak in the patient; administering orally a fifth oral dose in the amount of between about 3,000 micrograms to about 4,800 micrograms in the pharmaceutical dosage form during the prodromal phase of the herpes simplex virus outbreak in the patient; administering orally a sixth pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient; administering orally a seventh pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient; administering orally an eighth pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient; administering orally a ninth pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient; administering orally a tenth pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient; and suppressing the herpes simplex virus symptom in the patient during the prodromal phase of the herpes simplex virus outbreak in the patient.

In some embodiments of the invention the therapeutically effective pharmaceutical formulation of allicin in the pharmaceutical dosage form to the patient is administered over a time interval selected from the group consisting of hourly, daily, and a combination thereof. In some embodiments of the invention the oral pharmaceutical formulation of the aliicin is administered in the pharmaceutical dosage form selected from the group consisting of a capsule pharmaceutical dosage form, a tablet pharmaceutical dosage form, and a gelcap pharmaceutical dosage form, and a combination thereof. Some embodiments of the invention relate to methods of administering orally a therapeutically effective pharmaceutical formulation of between about 3000 micrograms to about 12,000 micrograms of allicin in a pharmaceutical dosage form to a patient with a herpes simplex viral infection (HSV-1 and/or HSV-2 infection), the therapeutically effective pharmaceutical formulation of allicin for increasing a human immune system activity in the patient during a reactivation of a herpes simplex virus causing a prodromal phase of the herpes simplex virus outbreak in the patient, the increasing of the human immune system activity in the patient for suppressing a herpes simplex viral symptom in the patient. In some embodiments, allicin doses are administered hourly or daily for a total of ten dose administrations or until a herpes simplex viral symptom is suppressed. Procedures for making capsules, tablets and gelcaps are well knonw in the art of pharmaceutical formulation and pharmaceutical dosage forms.

Certain terminology may be used in the following description for convenience only and is not limiting. The terminology includes the words above specifically mentioned, derivatives thereof and words of similar import. The use of the word “or” when stating for example the expression “A or B” is also intended to signify stating the expression “A and B.” However, the use of the word “and” when stating for example the expression “C and D” is not intended to signify stating the expression “C or D”. Where a term is provided in the singular, the inventors also contemplate aspects of the invention described by the plural of that term. As used in this specification and in the appended claims, the singular forms “a”, “an” and “the” include plural references unless the context clearly dictates otherwise, e.g., “a tip” includes a plurality of tips. Thus, for example, a reference to “a method” includes one or more methods, and/or steps of the type described herein and/or which will become apparent to those persons skilled in the art upon reading this disclosure. Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods, constructs and materials are now described. All publications mentioned herein are incorporated herein by reference in their entirety. Where there are discrepancies in terms and definitions used in references that are incorporated by reference, the terms used in this application shall have the definitions given herein.

It will be appreciated by those skilled in the art that changes could be made to the embodiments described above in writing, in a diagram, in a drawing, and in a figure without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A method of administering orally a therapeutically effective pharmaceutical formulation of allicin in a pharmaceutical dosage form to a patient with a herpes simplex viral infection, the therapeutically effective pharmaceutical formulation of allicin for increasing a human immune system activity in the patient during a reactivation of a herpes simplex virus causing a prodromal phase of the herpes simplex virus outbreak in the patient, the increasing of the human immune system activity in the patient for suppressing a herpes simplex viral symptom in the patient, the method comprising the steps of:
administering orally a first therapeutically effective pharmaceutical formulation of the allicin with a dose of the allicin in an amount of between about 7,200 micrograms to about 12,000 micrograms in the pharmaceutical dosage form during onset of the prodromal phase of the herpes simplex virus outbreak in the patient;
administering orally a second pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient;
administering orally a third pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient;
administering orally a fourth pharmaceutical formulation of the allicin with the dose of the allicin in the amount of between about 3,000 micrograms to about 4,800 micrograms in the pharmaceutical dosage form during the prodromal phase of the outbreak in the patient;
administering orally a fifth oral dose in the amount of between about 3,000 micrograms to about 4,800 micrograms in the pharmaceutical dosage form during the prodromal phase of the herpes simplex virus outbreak in the patient;
administering orally a sixth pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient;
administering orally a seventh pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient;
administering orally an eighth pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient;
administering orally a ninth pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient;
administering orally a tenth pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient; and
suppressing the herpes simplex virus symptom in the patient during the prodromal phase of the herpes simplex virus outbreak in the patient, and
wherein the administering orally of the therapeutically effective pharmaceutical formulation of allicin in the pharmaceutical dosage form to the patient occurs over a time interval selected from the group consisting of an hourly time interval, and a daily time interval, and a combination thereof.

2. The method according to claim 1, wherein the reactivation of the herpes simplex virus follows a period of latency in the patient,
wherein the herpes simplex virus resides in the trigeminal ganglion in the patient,
wherein the herpes simplex viral symptom in the patient is expressed on the face of the patient as a cold sore symptom of the herpes simplex virus, and
wherein the initial dose of the allicin and subsequent doses administered to the patient at timed intervals enable a suppression of the herpes simplex viral symptom in the patient which is expressed on the face of the patient as a cold sore symptom of the herpes simplex virus.

3. The method according to claim 1, wherein the reactivation of the herpes simplex virus follows a period of latency in the patient,
wherein the herpes simplex virus resides in the sacral ganglion in the patient,
wherein the herpes simplex viral symptom in the patient is expressed on a genital area of the patient as a genital sore symptom of the herpes simplex virus, and
wherein the initial dose of the allicin and subsequent doses administered to the patient at timed intervals enable a suppression of the herpes simplex viral symptom in the patient which is expressed in the genital area of the patient as a sore symptom of the herpes simplex virus.

4. The method according to claim 1, wherein the oral pharmaceutical formulation of the allicin with the dose of the allicin is in a capsule pharmaceutical dosage form.

5. The method according to claim 4, further comprising an additional ingredient selected from the group consisting of 10 mg lysine, a vanilla flavoring, a lemon verbena, and a combination thereof.

6. The method according to claim 1, wherein the oral pharmaceutical formulation of the allicin with the dose of the allicin is in the pharmaceutical dosage form selected from the group consisting of a tablet pharmaceutical dosage form, and a gelcap pharmaceutical dosage form.

7. The method according to claim 6, further comprising an additional ingredient selected from the group consisting of 10 mg lysine, a vanilla coating, a lemon verbena, and a combination thereof.

8. The method according to claim 2, wherein the oral pharmaceutical formulation of the allicin with the dose of the allicin is in a capsule pharmaceutical dosage form.

9. The method according to claim 8, wherein the oral pharmaceutical formulation of the allicin with the dose of the allicin is in a capsule pharmaceutical dosage form, and further comprises an additional ingredient selected from the group consisting of 10 mg lysine, a vanilla flavoring, a lemon verbena, and a combination thereof.

10. The method according to claim 2, wherein the oral pharmaceutical formulation of the allicin with the dose of the allicin is in the pharmaceutical dosage form selected from the group consisting of a tablet pharmaceutical dosage form, and a gelcap pharmaceutical dosage form.

11. The method according to claim 10, further comprising an additional ingredient selected from the group consisting of 10 mg lysine, a vanilla coating, a lemon verbena, and a combination thereof.

12. The method according to claim 3, wherein the oral pharmaceutical formulation of the allicin with the dose of the allicin is in a capsule pharmaceutical dosage form.

13. The method according to claim 12, wherein the oral pharmaceutical formulation of the allicin with the dose of the allicin is in a capsule pharmaceutical dosage form, and further comprises an additional ingredient selected from the group consisting of 10 mg lysine, a vanilla flavoring, a lemon verbena, and a combination thereof.

14. The method according to claim 3, wherein the oral pharmaceutical formulation of the allicin with the dose of the allicin is in the pharmaceutical dosage form selected from the group consisting of a tablet pharmaceutical dosage form, and a gelcap pharmaceutical dosage form.

15. The method according to claim 14, further comprising an additional ingredient selected from the group consisting of 10 mg lysine, a vanilla coating, a lemon verbena, and a combination thereof.

16. A method of administering orally a therapeutically effective pharmaceutical formulation of allicin in a pharmaceutical dosage form to a patient with a herpes simplex viral infection, the therapeutically effective pharmaceutical formulation of allicin for increasing a human immune system activity in the patient during a reactivation of a herpes simplex virus causing a prodromal phase of the herpes simplex virus outbreak in the patient, the increasing of the human immune system activity in the patient for suppressing a herpes simplex viral symptom in the patient, the method comprising the steps of:
administering orally a first therapeutically effective pharmaceutical formulation of the allicin with a dose of the allicin in an amount of between about 7,200 micrograms to about 12,000 micrograms during onset of the prodromal phase of the herpes simplex virus outbreak in the patient;
administering orally a second pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient;
administering orally a third pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient;
administering orally a fourth pharmaceutical formulation of the allicin with the dose of the allicin in the amount of between about 3,000 micrograms to about 4,800 micrograms in the pharmaceutical dosage form during the prodromal phase of the outbreak in the patient;
administering orally a fifth oral dose in the amount of between about 3,000 micrograms to about 4,800 micrograms in the pharmaceutical dosage form during the prodromal phase of the herpes simplex virus outbreak in the patient;
administering orally a sixth pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient;
administering orally a seventh pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient;
administering orally an eighth pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient;
administering orally a ninth pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient;
administering orally a tenth pharmaceutical formulation of the allicin with the dose of the allicin in the amount of between about 3,000 micrograms to about 4,800 micrograms in the pharmaceutical dosage form during the prodromal phase of the herpes simplex virus outbreak in the patient; and
suppressing the herpes simplex virus symptom in the patient during the prodromal phase of the herpes simplex virus outbreak in the patient.

17. The method according to claim 16, wherein the administering orally of the therapeutically effective pharmaceutical formulation of allicin in the pharmaceutical dosage form to the patient occurs over a time interval selected from the group consisting of hourly, daily, and a combination thereof.

18. The method according to claim 16, wherein the oral pharmaceutical formulation of the aliicin is administered in the pharmaceutical dosage form selected from the group consisting of a capsule pharmaceutical dosage form, a tablet pharmaceutical dosage form, and a gelcap pharmaceutical dosage form, and a combination thereof.

19. The method according to claim 16,
wherein the reactivation of the herpes simplex virus follows a period of latency in the patient,
wherein the herpes simplex virus resides in the trigeminal ganglion in the patient,
wherein the herpes simplex viral symptom in the patient is expressed on the face of the patient as a cold sore symptom of the herpes simplex virus, and
wherein the initial dose of the allicin and subsequent doses administered to the patient at timed intervals enable a suppression of the herpes simplex viral symptom in the patient which is expressed on the face of the patient as a cold sore symptom of the herpes simplex virus.

20. The method according to claim 18, further comprising an additional ingredient selected from the group consisting of 10 mg lysine, a vanilla ingredient, a lemon verbena, and a combination thereof.
